(19) United States
(12) Patent Application Publication
(10) Pub. No.: US 2026/0020746 A1
(43) Pub. Date: Jan. 22, 2026

(54) MEDICAL ASSEMBLIES, DEVICES, AND SYSTEMS HAVING A MICRO-ELECTROMECHANICAL SYSTEM

(71) Applicant: Boston Scientific Scimed, Inc., Maple Grove, MN (US)

(72) Inventors: Jeffrey C. MARRION, Littleton, MA (US); Vivek SHAH, Burlington, MA (US); Adam Michael LAROUCHE, Bloomington, IN (US); Niraj Prasad RAUNIYAR, Plymouth, MN (US); Preston STEELE, Franklin, MA (US); Camron HAGEMEYER, Bloomington, IN (US); Daniel J. FOSTER, Lino Lakes, MN (US); Anya S. CHASE, Dover, NH (US); Keith R. MAILE, New Brighton, MN (US); Daniel Frank MASSIMINI, Brooklyn Park, MN (US); Justin Theodore NELSON, Vadnais Heights, MN (US); Nathan GAWORSKI, St. Paul, MN (US)

(73) Assignee: Boston Scientific Scimed, Inc., Maple Grove, MN (US)

(21) Appl. No.: 19/266,903

(22) Filed: Jul. 11, 2025

Related U.S. Application Data
(60) Provisional application No. 63/671,891, filed on Jul. 16, 2024.

(51) Int. Cl. A61B 1/00; A61B 1/005

(52) U.S. Cl. CPC A61B 1/00097; A61B 1/00066; A61B 1/005; A61B 2560/0462; A61B 2562/0247; A61B 2562/0271; A61B 2562/028

(57) ABSTRACT

Medical devices are described, including a medical device including a shaft extending from a proximal end to a distal end portion. An outermost surface of the distal end portion may include an opening in fluid communication with an environment around the distal end portion, a housing including a cavity in fluid communication with the opening, and a micro-electromechanical system (MEMS) assembly including at least one sensor. The MEMS assembly may be disposed on a support within the cavity.

### CROSS-REFERENCE TO RELATED APPLICATIONS

This application claims the benefit of priority to U.S. Provisional Application No. 63/671,891, filed on Jul. 16, 2024, which is incorporated by reference herein in its entirety.

### TECHNICAL FIELD

Aspects of the present disclosure generally relate to medical assemblies, devices, and systems. In particular, some aspects relate to medical devices and systems having a micro-electromechanical system assembly incorporated in a distal portion of the medical device.

### BACKGROUND

Medical devices are often inserted into the body to perform a therapeutic and/or diagnostic procedure inside a subject's body. An example of such a device is an ureteroscope or other type of scope, which includes an insertion portion that is introduced into the body. Various features of the scope may assist in performing a therapeutic and/or diagnostic procedure inside the subject's body. Sensors and other electronic components may be susceptible to damage, e.g., due to external forces. Such forces can degrade performance over time or ultimately lead to failure of the electronic component(s).

### SUMMARY

Each of the aspects disclosed herein may include one or more of the features described in connection with any of the other disclosed aspects.

The present disclosure includes medical assemblies, devices, and systems useful for preserving the functionality of electronic components, such as microelectronic mechanical system assemblies, e.g., at the distal end and/or tip of the device.

According to some aspects of the present disclosure, the medical device may include a shaft extending from a proximal end to a distal end portion. An outermost surface of the distal end portion may include an opening in fluid communication with an environment around the distal end portion, a housing including a cavity in fluid communication with the opening, and a micro-electromechanical system (MEMS) assembly including at least one sensor. The MEMS assembly may be disposed on a support within the cavity.

According to some aspects, the housing may include a stepped portion that includes the support. The support may be a wall of the housing. The at least one sensor may face the opening. An entire length of the MEMS assembly may be attached to the support. The MEMS assembly may include a MEMS chip and the at least one sensor is disposed on a surface of the MEMS chip. The support may include a material having thermal properties similar to the MEMS assembly. Only a portion of the MEMS assembly may be disposed on the support. The at least one sensor may face away from the opening. The medical device may include a handle coupled to the shaft and the handle may include a processor operably coupled to the MEMS assembly. The support may extend along a plane transverse to a plane that includes a perimeter of the opening. The at least one sensor may face a direction perpendicular to the opening. The at least one sensor may be a pressure sensor that includes a diaphragm. A surface of the diaphragm may include at least one of a hydrophilic coating or a metallic coating. The at least one sensor may be configured to measure both pressure and temperature.

The present disclosure also includes, for example, a medical device including a handle and a shaft extending from the handle to a distal end portion. The distal end portion may include an opening in fluid communication with an environment around the shaft, a housing including a cavity in fluid communication with the opening, and a micro-electromechanical system (MEMS) assembly including at least one sensor. The MEMS assembly may be disposed within the housing and the at least one sensor may face away from the opening. The at least one sensor may be a pressure sensor. The MEMS assembly may be disposed on a support that comprises a ceramic.

The present disclosure also includes, for example, a medical device including a handle and a shaft extending from the handle to a distal end portion. The distal end portion may include an opening in fluid communication with an environment around the shaft and a housing including a cavity in fluid communication with the opening. The housing may include one or more supports. The distal end portion may further include a micro-electromechanical system (MEMS) assembly including a MEMS chip and at least one sensor disposed on a surface of the MEMS chip. The MEMS assembly may be disposed on the one or more supports. The housing may include a first support on a first end of the MEMS chip and a second support on a second end of the MEMS chip opposite the first end.

### BRIEF DESCRIPTION OF THE FIGURES

The accompanying drawings, which are incorporated in and constitute a part of this application, illustrate exemplary aspects of this disclosure and together with the description, serve to explain the principles of the present disclosure.

FIG. 1A illustrates an exemplary medical system including a medical device, in accordance with some aspects of this disclosure.

FIG. 1B illustrates a distal end of the medical device of FIG. 1A, in accordance with some aspects of this disclosure.

FIG. 1C illustrates an exemplary housing, in accordance with some aspects of this disclosure.

FIG. 1D illustrates features of an exemplary MEMS assembly within the housing of FIG. 1C proximate the distal end of the medical device of FIG. 1A, in accordance with some aspects of this disclosure.

FIG. 2 illustrates another exemplary housing, in accordance with some aspects of this disclosure.

FIGS. 3A and 3B illustrate a further exemplary housing, in accordance with some aspects of this disclosure.

FIG. 4 illustrates yet another exemplary housing, in accordance with some aspects of this disclosure.

FIG. 5 illustrates features of another exemplary MEMS assembly of a medical device, in accordance with some aspects of this disclosure.

FIG. 6 illustrates features of a further exemplary MEMS assembly of a medical device, in accordance with some aspects of this disclosure.

FIGS. 7A and 7B illustrate features of yet another exemplary MEMS assembly of a medical device, in accordance with some aspects of this disclosure.

FIG. 8 illustrates features of another exemplary MEMS assembly of a medical device, in accordance with some aspects of this disclosure.

### DETAILED DESCRIPTION

Particular aspects of the present disclosure are described in greater detail below. The terms and definitions provided herein control, if in conflict with terms and/or definitions incorporated by reference. Wherever possible, the same or similar reference numbers will be used through the drawings to refer to the same or like parts.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body. Proximal and distal directions are labeled with arrows marked “P” and “D,” respectively, throughout various figures.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Relative terms such as “about,” “substantially,” and “approximately,” etc., are used to indicate a possible variation of ±10% of the stated numeric value or range.

Although ureteroscopes are referenced herein for illustration purposes, it will be appreciated that the disclosure encompasses any suitable medical device configured to allow an operator to access and view internal body anatomy of a subject (e.g., patient) and/or to deliver medical instruments, such as, for example, biopsy forceps, graspers, baskets, snares, probes, scissors, retrieval devices, lasers, and other tools, into the subject's body. The medical devices herein may be inserted into a variety of body lumens and/or cavities, such as, for example, the urinary tract or gastrointestinal tract. It will be appreciated that, unless otherwise specified, bronchoscopes, duodenoscopes, endoscopes, gastroscopes, endoscopic ultrasonography (“EUS”) scopes, colonoscopes, laparoscopes, cystoscopes, aspiration scopes, sheaths, catheters, or any other suitable delivery device or medical device may be used in connection with the features described herein.

The present disclosure includes medical devices comprising at least one sensor. For example, the medical devices herein may comprise one or more fiber-optic sensors and/or micro-electromechanical system (MEMS) assembly useful for therapeutic and/or diagnostic procedures. The MEMS assemblies herein may provide a combination of mechanical and electrical or electronic functionality such as, e.g., one or more sensors, chips (e.g., integrated circuit chip(s)) and/or other circuitry, and/or structural features. For example, the fiber optic sensor(s) and/or MEMS assembly may collect information regarding surrounding anatomy, e.g., a target organ, of a subject to assist a medical professional to determine an appropriate treatment. The MEMS assemblies herein may help to navigate the medical device during a procedure, e.g., to avoid or prevent damage to tissue such as perforation or bleeding in the procedure.

As mentioned above, a MEMS assembly may be subjected to a variety of external forces that may damage one or more sensors and/or other components or aspects of the MEMS assembly. For example, the MEMS assembly may be subjected to external forces during insertion, removal and/or use. The forces may be a result of the MEMS assembly colliding with, or abutting against, the subject's tissue. The forces may also be a result of a lithotripsy procedure or other medical procedure that produces energy, e.g., shockwaves. Damage to the MEMS assembly may result in increased procedural times, decreased accuracy of measurements, and/or loss of therapeutic or diagnostic functionality. In an example in which the MEMS assembly includes a pressure sensor, damage to the MEMS assembly may result in inaccurate pressure measurements and may prevent the medical device from indicating to a medical professional that the pressure at a target site may pose risks to the patient, e.g., the pressure being too high.

According to some aspects of the present disclosure, a medical device may include a MEMS assembly with features useful for inhibiting or preventing damage thereto, among other aspects. The MEMS assembly may be integrated into, onto, or within a distal end portion of a medical device, such as a ureteroscope. The MEMS assembly may include a MEMS chip and a sensor, e.g., disposed on a surface of the MEMS chip. The sensor may be exposed to an external environment surrounding the distal end of the medical device via an opening in an outer surface of the distal end portion of the medical device (e.g., a lateral opening in the shaft or end cap of the medical device proximate the distal end). The sensor may be utilized, for example, to provide data associated with a therapeutic or diagnostic procedure performed inside the subject's body.

According to some aspects of the present disclosure, the MEMS assembly may be disposed on, e.g., at least partially mounted on or otherwise attached to, one or more supports proximate the distal end portion of the medical device. The one or more supports may extend adjacent to a surface of the MEMS assembly (e.g., attached to an entire length of the MEMS assembly or ends of the MEMS assembly) and may help to buttress the MEMS assembly, e.g., against external forces during a medical procedure. In some examples, one or more supports may be disposed on opposite sides of the MEMS assembly and the one or more supports may help prevent and/or reduce movement of the MEMS assembly, e.g., during a medical procedure. In some examples, the sensor may face the opening of the distal end portion of the medical device. In other examples, the sensor may face away from the opening of the distal end portion of the medical device while still being able to collect data relating to the external environment surrounding the distal end of the medical device. According to some aspects of the present disclosure, a material may be provided on and/or surround the MEMS assembly to further protect the MEMS assembly.

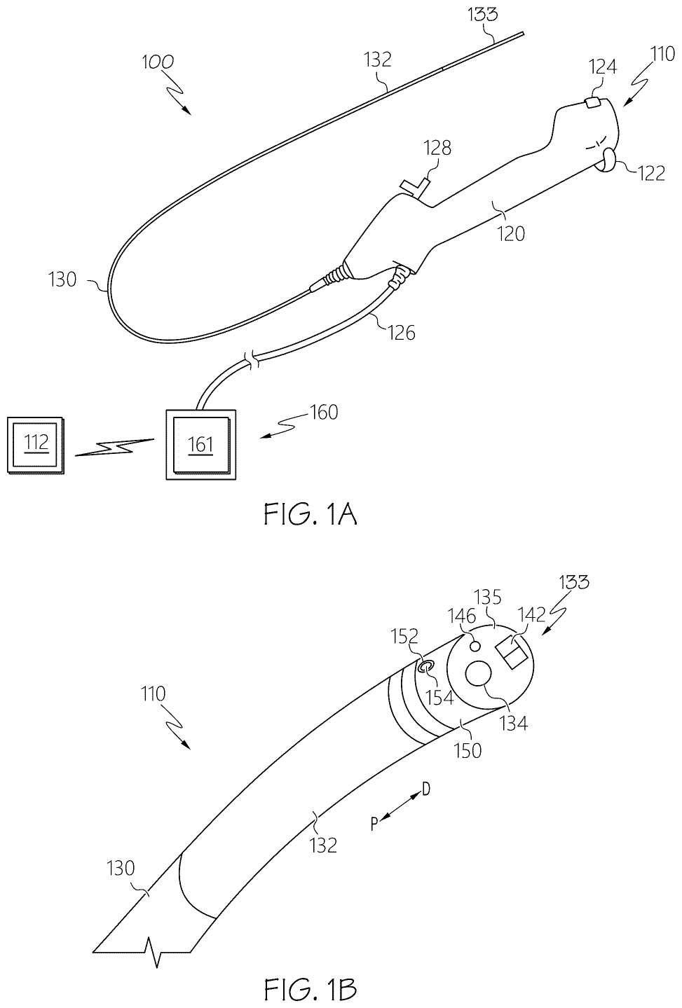

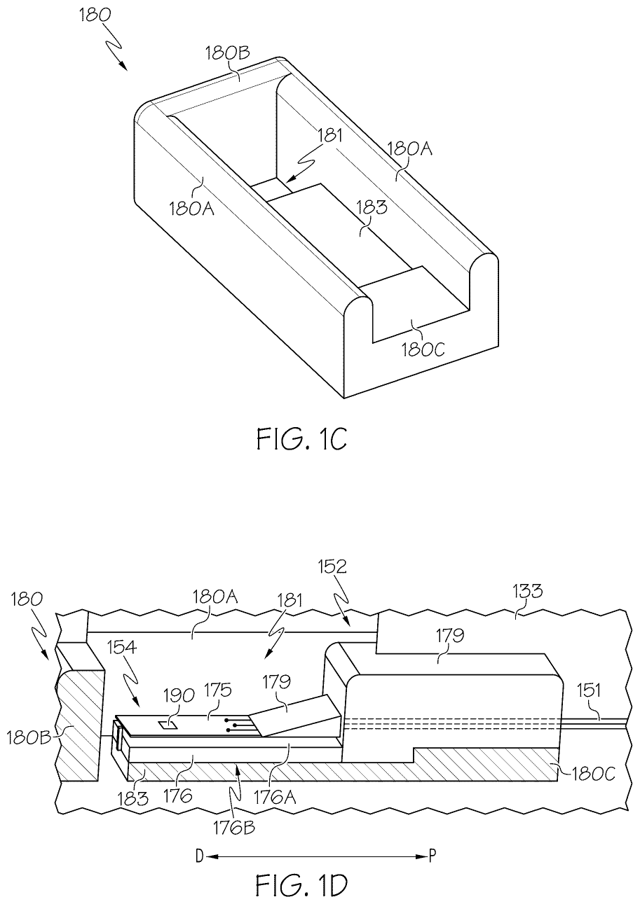

An exemplary medical system 100 is now described with reference to FIG. 1A. System 100 comprises a medical device 110, e.g., a ureteroscope, which may be operably coupled to equipment 160 supporting medical device 110. Medical device 110 may include a handle 120 with at least one actuator, e.g., a first actuator 122 and a second actuator 124, a port 128, and a shaft 130 with a steerable portion 132. Shaft 130 may extend to a distal tip portion 133. In some examples, distal tip portion 133 may be integral with shaft 130. In other examples, distal tip portion 133 may be a separate piece coupled to shaft 130. For example, distal tip portion 133 may comprise an end cap fixedly or detachably coupled to the distal end of shaft 130.

Actuators 122, 124 may receive user input and transmit the user input to shaft 130. Each actuator 122, 124 may include a lever, knob, slider, joystick, button, or other suitable mechanism. For example, first actuator 122 may include a lever configured to articulate steerable portion 132, e.g., via one or more pull wires within shaft 130, and second actuator 124 may include a button configured to actuate and/or control other aspects of medical device 110, e.g., turning on/off light sources and/or controlling an imaging device to capture images.

Equipment 160 may be configured to supply medical device 110 with vacuum/suction, fluid (e.g., liquid, air), and/or power via an umbilicus 126. As shown in FIG. 1A, equipment 160 may include a processing unit 161 (alternatively referred to as a processor), e.g., operable with medical device 110. For example, processing unit 161 may help generate a visual representation of image data and/or transmit the visual representation to one or more interface devices, e.g., a display 112. Display 112 may include, e.g., a touch-screen display, capable of displaying images captured using medical device 110 and processing unit 161. Processing unit 161 may be configured to receive signals and/or data from one or more sensors of medical device 110, which will be discussed in greater detail below. Alternatively, processing unit 161 may be incorporated into medical device 110. For example, processing unit 161 may be included in handle 120.

As mentioned above, umbilicus 126 may operably couple medical device 110 with equipment 160. Umbilicus 126 may sheath various cables and wirings for electric/electronic connection and/or one or more tubes for providing fluidics to medical device 110 (e.g., to distal tip portion 133). In aspects, umbilicus 126 may include one or more lumens, for example, to contain and/or separate the various cables, wirings, and/or tubes.

Port 128 may include one or more openings in communication with a working channel 134 of shaft 130. While port 128 is illustrated in this example on a distal portion of handle 120, port 128 may disposed on or incorporated to other portions of handle 120. A suitable accessory instrument or tool, e.g., a fiber (e.g., laser fiber), grasper, retrieval device, etc., may be inserted through port 128 and moved distally through shaft 130 via working channel 134. Shaft 130 may further include one or more lumen(s) for receiving pull wires and/or other wiring, cables, and/or fluidics tubing.

As shown in FIG. 1B, medical device 110 may include imaging components such as an imaging device 142 (e.g., camera, imager, etc.) and one or more light sources 146 (e.g., LEDS, fiber optics, etc.) at distal tip portion 133. Working channel 134 of shaft 130 may terminate at an opening on a distalmost face 135 of distal tip portion 133. Distal tip portion 133 may include an opening 152 in an outermost surface 150 of distal tip portion 133, e.g., adjacent and/or proximal of distalmost face 135 of distal tip portion 133. For example, opening 152 may be a side-facing or lateral opening on a radially outer side of distal tip portion 133. Alternatively, opening 152 may be in distalmost face 135 of distal tip portion 133.

Medical device 110 may include or contain a MEMS assembly 154. For example, MEMS assembly 154 may be disposed within distal tip portion 133 and may be exposed to an external environment e.g., patient anatomy surrounding distal tip portion 133, via opening 152. Opening 152 may be any suitable shape or size to provide for adequate fluid communication to expose MEMS assembly 154 to the environment surrounding distal tip portion 133.

MEMS assembly 154 may include one or more sensors and a MEMS chip 176, wherein the one or more sensors may be disposed on or otherwise integrated with a surface of MEMS chip 176. The sensor(s) may be configured to measure one or more parameters associated with the external environment, e.g., pressure, temperature, pH, light, etc., and/or other parameters such as the position of MEMS assembly 154 relative to the external environment. The sensor(s) may include, for example, a pressure sensor, a temperature sensor, a humidity sensor, an accelerometer, a light sensor, a chemical sensor, a load cell, a pH sensor, and/or any other sensor useful in performing a therapeutic and/or diagnostic procedure. As shown in the example illustrated in FIG. 1D, MEMS assembly 154 may include a pressure sensor 175 on a first surface 176A of MEMS chip 176, wherein pressure sensor 175 may include a diaphragm 190 (e.g., an active portion or sensing element). In some examples, pressure sensor 175 may be configured to measure pressure and temperature in the environment surrounding distal tip portion 133.

One or more wires or cables 151 may extend proximally from MEMS assembly 154, through shaft 130, and into handle 120, e.g., to operably couple MEMS assembly 154 to handle 120 for receiving power and/or user input to control MEMS assembly 154. Wire(s)/cable(s) 151 may be electrically connected (directly or indirectly) to processing unit 161 and/or to other aspects of medical system 100. For example, wire(s)/cable(s) 151 may be configured to send signals and/or data from pressure sensor 175 to processing unit 161.

MEMS assembly 154 may be disposed in distal tip portion 133 within a housing 180 (e.g., a carrier), an example of which is illustrated in FIG. 1C. Housing 180 may assist with mounting MEMS assembly 154 within distal tip portion 133, among other aspects. Housing 180 may include a cavity 181 (e.g., a space) defined by walls and/or other surfaces of housing 180. Housing 180 may include a plurality of walls, e.g., two parallel walls 180A joined by a distal wall 180B and a proximal wall 180C. For example, housing 180 may have a substantially rectangular shape. Housing 180 may include a support 183, e.g., the support being provided by a wall of housing 180 or coupled to a wall of housing 180. Support 183 may extend from proximal wall 180C and between parallel walls 180A. Housing 180 may include a stepped portion, e.g., including proximal wall 180C and support 183. Housing 180 may be sized and shaped to be received within distal tip portion 133 such that cavity 181 is in fluid communication with opening 152.

As shown in FIG. 1D, MEMS assembly 154 may be disposed within cavity 181 of housing 180. FIG. 1D illustrates a partial cross-sectional view of housing 180 with one of the two parallel walls 180A removed to facilitate viewing components of MEMS assembly 154. MEMS assembly 154 may be oriented within cavity 181 such that diaphragm 190 of pressure sensor 175 faces opening 152. For example, MEMS assembly 154 may be proximate opening 152 of distal tip portion 133, wherein first surface 176A of MEMS chip 176, including pressure sensor 175, may extend along a plane parallel to a plane that includes a perimeter of opening 152. Pressure sensor 175, including diaphragm 190, may face away from a central longitudinal axis of distal tip portion 133.

In some aspects, an entire length of MEMS assembly 154 may be attached to support 183 as illustrated in the example shown in FIG. 1D. For example, a planar surface of support 183 may contact a second surface 176B of MEMS chip 176, second surface 176B being opposite first surface 176A. It will be appreciated that support 183 may have dimensions sufficient to support an entirety or a substantial portion of MEMS assembly 154. Support 183 may help to absorb forces on distal tip portion 133 during a medical procedure to reduce the risk of damage to MEMS assembly 154 from such forces or bending loads, among other aspects.

Support 183 in contact with MEMS assembly 154 may comprise a material having thermal properties similar to MEMS assembly 154, e.g., similar to pressure sensor 175 and/or MEMS chip 176. Having similar thermal properties may help to avoid differing responses to changes in temperature (e.g., from use of a laser fiber) during a medical procedure, wherein differing responses may risk degradation of the integrity of MEMS assembly 154. In some examples, the support may have a relatively low coefficient of thermal expansion. For example, support 183 may comprise a ceramic material.

As mentioned above, wire(s)/cable(s) 151 may extend proximally from MEMS assembly 154, e.g., to transmit data to processing unit 161. Wire(s)/cable(s) 151 may be in electronic communication with MEMS assembly 154 and fixed to or otherwise attached to MEMS assembly 154 with the assistance of an adhesive 179. Adhesive 179 may also assist with fixing or otherwise attaching wire(s)/cable(s) 151 and MEMS assembly 154 to housing 180. Examples of suitable adhesives include, but are not limited to, epoxy, acrylates (e.g., cyanoacrylate, acrylic), urethane, silicone, and hot-melt adhesives.

According to some aspects of the present disclosure, a hydrophobic material (e.g., silicone or hydrocarbon-based polymers, waxes, greases, oils) may be provided within cavity 181, e.g., to protect MEMS assembly 154 while permitting the transfer of force for measuring pressure. For example, at least a portion or an entirety of cavity 181 may be filled with the hydrophobic material. The hydrophobic material may have a viscosity sufficient to stay within cavity 181 and remain intact in the presence of bodily fluids. It will be appreciated that the hydrophobic material may be used with any of the MEMS assemblies and housings described herein.

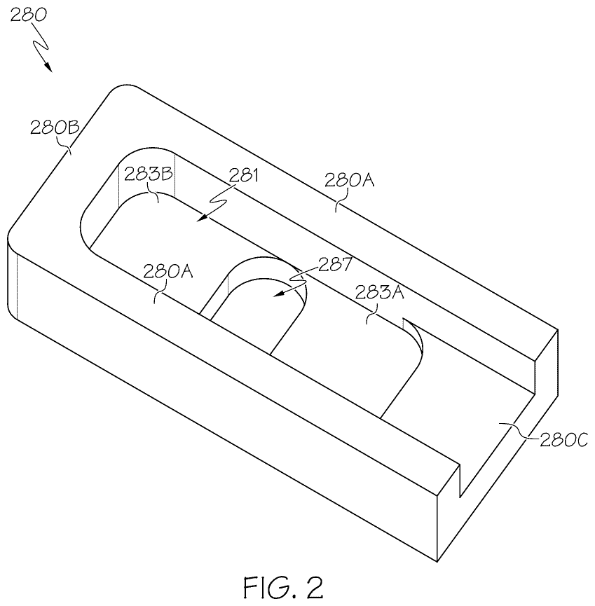

FIGS. 2, 3A, 3B, and 4 illustrate various exemplary housings that may be used with MEMS assembly 154 (e.g., in place of housing 180) and with any of the other MEMS assemblies described herein.

FIG. 2 illustrates an exemplary housing 280 having any of the features of housing 180, unless otherwise specified herein. Housing 280 may include a cavity 281, two parallel walls 280A, a distal wall 280B, and a proximal wall 280C. Housing 280 may include at least one support, optionally two supports (or one support divided into two portions). As illustrated in FIG. 2, housing 280 includes first and second supports 283A, 283B, which may have any of the features of support 183, unless otherwise specified herein. First support 283A may extend from proximal wall 280C and between parallel walls 280A. Second support 283B may extend from distal wall 280B and between parallel walls 280A, and a gap 287 may be between first and second supports 283A, 283B. When MEMS assembly 154 is disposed within housing 280, the proximal end of MEMS assembly 154 may be disposed on first support 283A, the distal end of MEMS assembly 154 may be disposed on second support 283B, and remaining portions of MEMS assembly 154 may extend over gap 287. Housing 280 may include a stepped portion, e.g., including proximal wall 280C and support 283A.

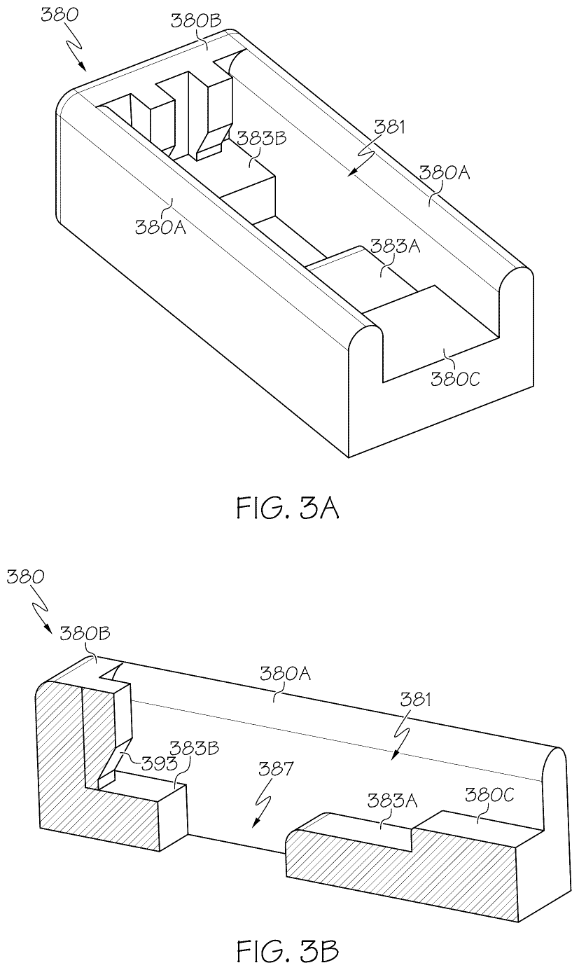

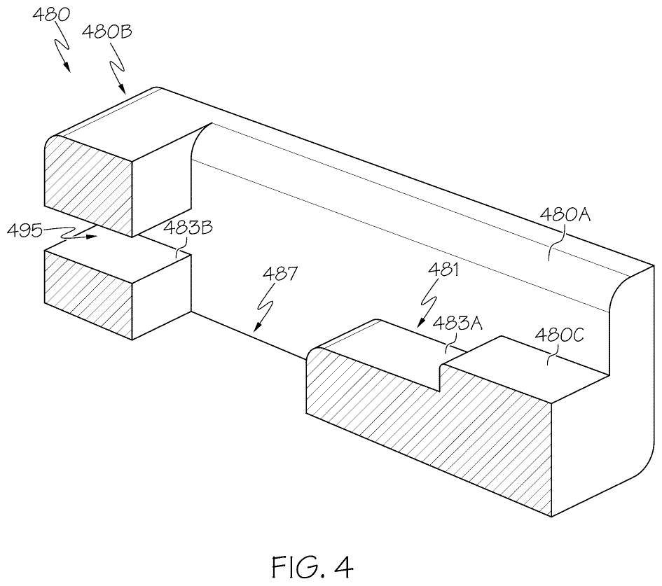

FIG. 3A and FIG. 3B (cross-sectional view of FIG. 3A) illustrate another exemplary housing 380 that may have any of the features of housings 180, 280, unless otherwise specified herein. Housing 380 may include a cavity 381, two parallel walls 380A, a distal wall 380B, and a proximal wall 380C. Housing 380 may include at least one support, optionally two supports (or one support divided into two portions). As illustrated in FIGS. 3A and 3B, housing 380 includes first and second supports 383A, 383B similar to first and second supports 283A, 283B, respectively. One or more portions of distal wall 380B of housing 380 may extend inward into cavity 381. Distal wall 380B may include a tapered surface 393 proximate second support 383B. Housing 380 may include a stepped portion, e.g., including proximal wall 380C and support 383A. When MEMS assembly 154 is disposed within housing 380, the proximal end of MEMS assembly 154 may be supported by first support 383A and the distal end of MEMS assembly 154 may be supported by second support 383B proximate tapered surface 393 of distal wall 380B. That is, the distal end of MEMS assembly 154 may be disposed between tapered surface 393 and second support 383B. Remaining portions of MEMS assembly 154 may extend over a gap 387 between first and second supports 383A, 383B. Distal wall 380B, including tapered surface 393, may assist with supporting MEMS assembly 154, e.g., to help prevent or reduce movement of MEMS assembly 154 during a medical procedure.

FIG. 4 illustrates a cross-sectional view of another exemplary housing 480, which may have any of the features of housings 180, 280, 380, unless otherwise specified herein. Housing 480 may include a cavity 481, two parallel walls 480A, a distal wall 480B, a proximal wall 480C, a first support 483A, and a second support 483B. First and second supports 483A, 483B may have any of the features of second supports 383A, 383B, unless otherwise specified herein. In this example, distal wall 480B defines second support 483B and an opening 495 capable of receiving an end of MEMS assembly 154. When MEMS assembly 154 is disposed within housing 480, the proximal end of MEMS assembly 154 may be disposed on first support 483A and the distal end of MEMS assembly 154 may be disposed on second support 483B within opening 495. Remaining portions of MEMS assembly 154 may extend over a gap 487 between first and second supports 483A, 483B. Similar to tapered surface 393, opening 495 may assist with supporting MEMS assembly 154, e.g., to help prevent or reduce movement of the MEMS assembly during a medical procedure.

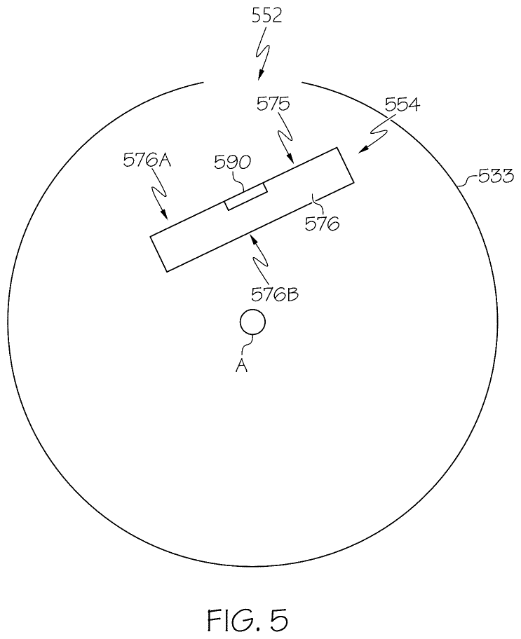

FIG. 5 illustrates a schematic view of an exemplary MEMS assembly 554 within a distal tip portion 533 of a medical device. MEMS assembly 554 may have any of the features of MEMS assembly 154, and distal tip portion 533 may have any of the features of distal tip portion 133. MEMS assembly 554 may be capable of measuring pressure of the external environment via an opening 552 in an outermost surface of distal tip portion 533. MEMS assembly 554 may include one or more sensors disposed on or otherwise integrated with a first surface 576A of a MEMS chip 576, the first surface 576A being opposite a second surface 576B of MEMS chip 576. For example, MEMS assembly 554 may include a pressure sensor 575 including a diaphragm 590 (e.g., an active portion or sensing element). MEMS assembly 554 may be used with housings 180, 280, 380, 480 described above so long as pressure sensor 575 of MEMS assembly 554 is in fluid communication with opening 552.

In this example illustrated in FIG. 5, pressure sensor 575 of MEMS assembly 554 generally faces opening 552 but is offset from opening 552. For example, an axis extending through a center of diaphragm 590 and perpendicular to a longitudinal axis of MEMS assembly 554 intersects a wall of distal tip portion 533 proximate opening 552. As shown in FIG. 5, pressure sensor 575, including diaphragm 590, faces opening 552, away from a central longitudinal axis A of distal tip portion 533. In other examples, pressure sensor 575 may be fully aligned with opening 552, e.g., an axis extending through a center of diaphragm 590 and perpendicular to a longitudinal axis of MEMS assembly 554 extends through a central area of opening 552. A pressure sensor 575 that faces opening 552 may be rotated from about 1 degree to about 45 degrees, e.g., from about 5 degrees to about 30 degrees, or from about 10 degrees to about 20 degrees, relative to an orientation in which pressure sensor 575 is fully aligned with opening. In some other examples, pressure sensor 575 may face away from opening 552 (see, e.g., FIG. 8).

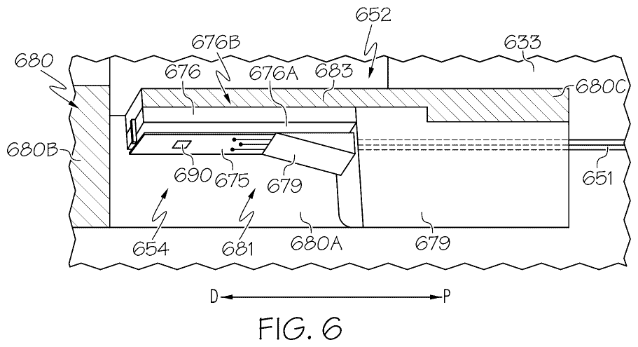

FIG. 6 illustrates another exemplary MEMS assembly 654 according to some aspects of the present disclosure. MEMS assembly 654 is shown disposed within a housing 680 in a distal tip portion 633 of a medical device. Distal tip portion 633 may have any of the features of distal tip portions 133, 533 discussed above; MEMS assembly 654 may have any of the features of MEMS assemblies 154, 554 discussed above; and housing 680 may have any of the features of housings 180, 280, 380, 480 discussed above, unless otherwise specified herein. MEMS assembly 654 is disposed proximate an opening 652 of distal tip portion 633 that provides fluid communication between MEMS assembly 654 and the external environment. In this example, a pressure sensor 675 of MEMS assembly 654 faces away from opening 652, e.g., housing 680 and MEMS assembly 654 being rotated about 180 degrees relative to the orientation of housing 180 and MEMS assembly 154 illustrated in FIG. 1D.

MEMS assembly 654 includes one or more sensors disposed on or otherwise integrated with a first surface 676A of a MEMS chip 676. For example, as mentioned above, MEMS assembly 654 may include a pressure sensor 675; optionally pressure sensor 675 is configured to measure temperature in addition to measuring pressure. Housing 680 may include a cavity 681 between two parallel walls 680A, a distal wall 680B, and a proximal wall 680C. Housing 680 may include a support 683 similar to support 183, e.g., an entire length of MEMS assembly 654 being disposed on support 683 (e.g., a second surface 676B of MEMS chip 676 disposed on support 683). One or more wires or cables 651 similar to wire(s)/cable(s) 151 may extend proximally from MEMS assembly 654. Adhesive 679 similar to adhesive 179 may assist with attaching wire(s)/cable(s) 651 to MEMS assembly 654 and/or attaching wire(s)/cable(s) 651/MEMS assembly 654 to housing 680.

As shown in FIG. 6, MEMS assembly 654 may be disposed within cavity 681 of housing 680 that is in fluid communication with opening 652 of distal tip portion 633. MEMS assembly 654 may be disposed within cavity 681 such that a diaphragm 690 (e.g., an active portion or sensing element) of pressure sensor 675 may face away from opening 652, e.g., in a direction opposite from opening 652 of distal tip portion 633. First surface 676A of MEMS chip 676, including pressure sensor 675, may face inward, towards a central longitudinal axis of distal tip portion 633. Support 683 extending along second surface 676B of MEMS chip 676 may help to dampen forces exposed to MEMS assembly 654, e.g., during a medical procedure. For example, support 683 may help to shield MEMS assembly 654 from shockwaves generated by a laser fiber and passing through distal tip portion 633. In some examples, support 683 covers only a portion of MEMS assembly 654, or MEMS assembly 654 does not include a support. In these instances, second surface 676B of MEMS chip 676 may face opening 652 of distal tip portion 633 and may help to dampen or otherwise shield diaphragm 690 from forces. It will be appreciated that MEMS assembly 654 may be used with housings 180, 280, 380, 480 described above so long as MEMS assembly 654 is in fluid communication with opening 652.

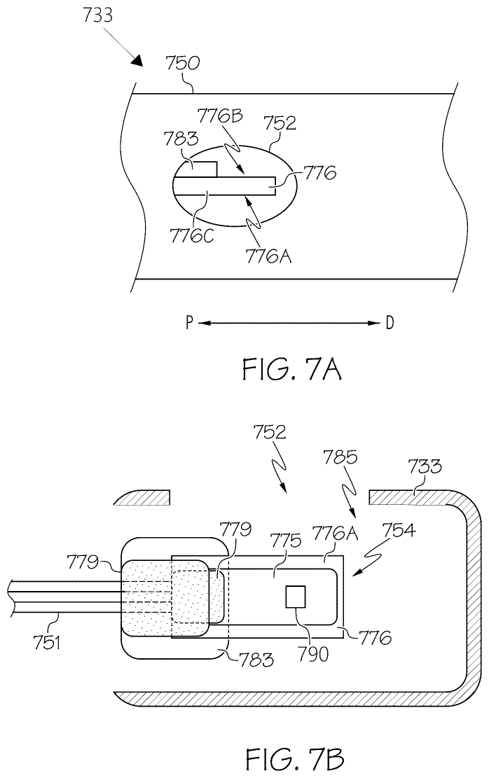

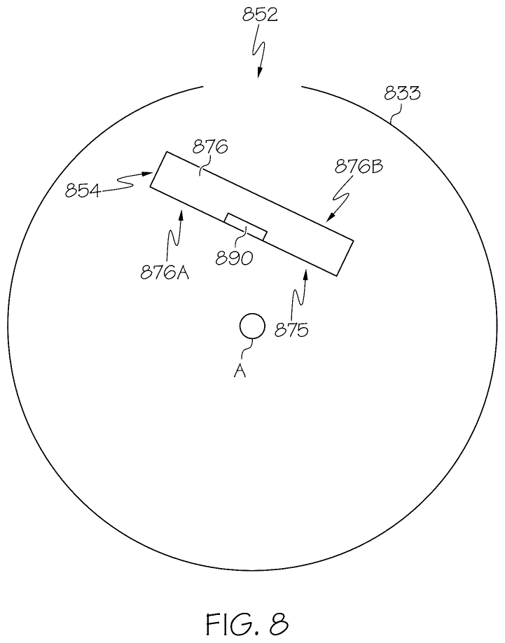

FIGS. 7A and 7B illustrate an exemplary MEMS assembly 754 within a distal tip portion 733. Distal tip portion 733 may have any of the features of distal tip portions 133, 533, 633; and MEMS assembly 754 may have any of the features of MEMS assemblies 154, 554, 654, unless otherwise specified herein. MEMS assembly 754 may include one or more sensors disposed on or otherwise integrated with a first surface 776A of a MEMS chip 776. For example, MEMS assembly 754 may include a pressure sensor 775 configured to measure pressure and/or temperature. One or more wires or cables 751 similar to wire(s)/cable(s) 151, 651 may be coupled to MEMS assembly 754 for transmitting data to processing unit 161.

In this example, MEMS assembly 754 is rotated about 90 degrees relative to the orientation of MEMS assembly 154 shown in FIG. 1D. As shown in FIGS. 7A and 7B, MEMS assembly 754 may be oriented perpendicular to an opening 752 in an outermost surface 750 of distal tip portion 733. MEMS assembly 754 may be disposed and/or mounted within a cavity 785 of distal tip portion 733 that is in fluid communication with opening 752 of distal tip portion 733. In some examples, MEMS assembly 754 may be disposed within a housing sized and shaped to be received within cavity 785 of distal tip portion 733. For example, MEMS assembly 754 may be used with housings 180, 280, 380, 480, 680 described above so long as MEMS assembly 754 is in fluid communication with opening 752.

FIG. 7B illustrates a cross-sectional side view of cavity 785 of distal tip portion 733. Adhesive 779 similar to adhesives 179, 679 may assist with attaching wires(s)/cable(s) 751 to MEMS assembly 754 and/or attaching wire(s)/cable(s) 751/MEMS assembly 754 to a support 783 within cavity 785. In some examples, support 783 may be a part of the housing. As shown in FIG. 7B, only a proximal end of MEMS chip 776 may be attached to support 783. In some examples, support 783 may extend along an entire length of MEMS assembly 754.

As mentioned above, MEMS assembly 754 may be oriented perpendicular to opening 752. First surface 776A of MEMS chip 776 including pressure sensor 775, and a second surface 776B of MEMS chip 776 may each face an inner surface of distal tip portion 733, in opposite directions. For example, first and second surfaces 776A, 776B of MEMS chip 776 may face away from opening 752 in a direction perpendicular to opening 752, and a side edge 776C of MEMS chip 776 may face towards opening 752. Similar to the configuration of MEMS assembly 654 in FIG. 2, the configuration of MEMS assembly 754 may help to protect pressure sensor 775 from forces (e.g., shockwaves) generated during a medical procedure.

FIG. 8 illustrates a schematic view of an exemplary MEMS assembly 854, which may have any of the features of MEMS assemblies 154, 554, 654, 754 described above. MEMS assembly 854 is disposed within a distal tip portion 833 of a medical device, distal tip portion 833 optionally having any of the features of distal tip portions 133, 533, 633, 733 described above. MEMS assembly 854 may be in fluid communication with the external environment of the medical device via an opening 852 in an outermost surface of distal tip portion 833. MEMS assembly 854 may include one or more sensors disposed on or otherwise integrated with a first surface 876A of a MEMS chip 876, first surface 876A being opposite a second surface 876B of MEMS chip 876. For example, MEMS assembly 854 may include a pressure sensor 875 including a diaphragm 890 (e.g., an active portion or sensing element). MEMS assembly 854 may be used with housings 180, 280, 380, 480, 680 described above so long as MEMS assembly 854 is in fluid communication with opening 852.

In this example, MEMS assembly 854 may be rotated relative to the orientation of MEMS assembly 154 illustrated in FIG. 5. As shown in FIG. 8, pressure sensor 875, including diaphragm 890 may face away from opening 852.

According to some aspects of the present disclosure, diaphragms 190, 590, 690, 790, 890 of sensors 175, 575, 675, 775, 875 may include various features to promote durability. For example, an increased thickness of the diaphragms, a material of the diaphragms, and/or a coating applied to the diaphragms may increase the strength of the sensors. These features may prevent and/or reduce damage to the sensors due to, e.g., shockwaves hitting the sensors. In some examples, a thickness of each of the diaphragms may be greater than approximately 2.0 μm, e.g., ranging from about 2.0 to about 5.0 μm.

As mentioned above, a coating may be applied to the diaphragms herein. The coating may comprise a metal, metal alloy, metal oxide, metal nitride, metal carbide, metal fluoride, metal silicide, and/or metal sulfide. Exemplary metals of the coating herein may include, e.g., gold, tantalum, molybdenum, silicon, aluminum, copper, silver, titanium, carbon, indium, hafnium, palladium, tungsten, zirconium, zinc, vanadium, tin, nickel, iron, germanium, niobium, manganese, cobalt, magnesium, and/or chromium, and alloys, oxides, nitrides, carbides, fluorides, silicides, and sulfides thereof. A metallic coating may increase the stiffness of the diaphragm and/or increase resiliency, e.g., to withstand external forces without damage. A metallic coating may change the resonance frequency of the diaphragm. For example, the metal(s) may improve durability by reducing vibration of the diaphragm when encountering a shockwave.

In some examples, the coating may comprise a hydrophilic material. The coating may be devoid of air bubbles. The coating may comprise a surfactant (e.g., Silsurf® B608, Tween®), polyethylene glycol (PEG), polyvinyl alcohol (PVA), polyacrylic acid (PAA), and/or polyvinylpyrrolidone (PVP). In some examples, the coating may comprise a hydrogel comprising, for example, gelatin, PEG, PVA, PAA, and/or PVP. It will be appreciated that the coatings described herein may be applied to other portions of the sensor in addition to the diaphragm and/or to surfaces of the housings and/or the distal tip portions described herein.

It will be apparent to those skilled in the art at various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

What is claimed is:

1. A medical device comprising:
a shaft extending from a proximal end to a distal end portion, wherein an outermost surface of the distal end portion includes:
an opening in fluid communication with an environment around the distal end portion;
a housing including a cavity in fluid communication with the opening; and
a micro-electromechanical system (MEMS) assembly including at least one sensor, wherein the MEMS assembly is disposed on a support within the cavity.

2. The medical device of claim 1, wherein the housing comprises a stepped portion that includes the support.

3. The medical device of claim 1, wherein the support is a wall of the housing.

4. The medical device of claim 1, wherein the at least one sensor faces the opening.

5. The medical device of claim 1, wherein an entire length of the MEMS assembly is attached to the support.

6. The medical device of claim 1, wherein the MEMS assembly includes a MEMS chip and the at least one sensor is disposed on a surface of the MEMS chip.

7. The medical device of claim 1, wherein the support comprises a material having thermal properties similar to the MEMS assembly.

8. The medical device of claim 1, wherein only a portion of the MEMS assembly is disposed on the support.

9. The medical device of claim 1, wherein the at least one sensor faces away from the opening.

10. The medical device of claim 1, further comprising a handle coupled to the shaft, wherein the handle includes a processor operably coupled to the MEMS assembly.

11. The medical device of claim 1, wherein the support extends along a plane transverse to a plane that includes a perimeter of the opening.

12. The medical device of claim 11, wherein the at least one sensor faces a direction perpendicular to the opening.

13. The medical device of claim 1, wherein the at least one sensor is a pressure sensor that includes a diaphragm.

14. The medical device of claim 13, wherein a surface of the diaphragm includes at least one of a hydrophilic coating or a metallic coating.

15. The medical device of claim 13, wherein the at least one sensor is configured to measure both pressure and temperature.

16. A medical device comprising:
a handle;
a shaft extending from the handle to a distal end portion, wherein the distal end portion includes:
an opening in fluid communication with an environment around the shaft;
a housing including a cavity in fluid communication with the opening; and
a micro-electromechanical system (MEMS) assembly including at least one sensor, wherein the MEMS assembly is disposed within the housing, and wherein the at least one sensor faces away from the opening.

17. The medical device of claim 16, wherein the at least one sensor is a pressure sensor.

18. The medical device of claim 16, wherein the MEMS assembly is disposed on a support that comprises a ceramic.

19. A medical device comprising:
a handle;
a shaft extending from the handle to a distal end portion, wherein the distal end portion includes:
an opening in fluid communication with an environment around the shaft;
a housing including a cavity in fluid communication with the opening, wherein the housing includes one or more supports; and
a micro-electromechanical system (MEMS) assembly including a MEMS chip and at least one sensor disposed on a surface of the MEMS chip, wherein the MEMS assembly is disposed on the one or more supports.

20. The medical device of claim 19, wherein the housing includes a first support on a first end of the MEMS chip and a second support on a second end of the MEMS chip opposite the first end.
